(19) United States
(12) Patent Application Publication
(10) Pub. No.: US 2026/0096951 A1
(43) Pub. Date: Apr. 9, 2026

(54) CANCER THERAPY DELIVERY SYSTEM WITH TUMBLING CONTAINER FOR SUSPENSION GENERATION

(71) Applicant: Boston Scientific Scimed, Inc., Maple Grove, MN (US)

(72) Inventors: Carl Parent, Brooklyn Park, MN (US); Patrick A. Haverkost, Crystal, MN (US); Robert M. Wold, Maple Lake, MN (US); Kianna Y. Elahi-Gedwillo, Minneapolis, MN (US); Scott McGhee, Ottawa (CA); David Copeland, Minnetonka, MN (US); Burke Minahan, Minneapolis, MN (US); Greg Kramer, Minneapolis, MN (US); Madeleine Berkson, Cambridge, MA (US); Dallas Erdahl, Minneapolis, MN (US)

(21) Appl. No.: 19/354,493

(22) Filed: Oct. 9, 2025

Related U.S. Application Data
(60) Provisional application No. 63/705,383, filed on Oct. 9, 2024.

(51) Int. Cl. A61J 1/16; A61J 1/06; A61J 1/14; A61K 51/12; A61N 5/10

(52) U.S. Cl. CPC A61J 1/16; A61J 1/065; A61J 1/1412; A61J 1/1475; A61K 51/1251; A61N 5/1007; A61K 2121/00; A61N 2005/1021

(57) ABSTRACT

Embodiments herein relate to systems for generating therapeutic suspensions of microspheres and carrier fluid that include a tumbling or rotating container. In an embodiment, a cancer therapy delivery system is included having a tumbler vial, the tumbler vial defining an interior volume. The system also includes a rotation mechanism configured to rotate the tumbler vial, a fluid inlet in fluid communication with the interior volume, a fluid outlet in fluid communication with the interior volume, a fluid inlet line connected to the fluid inlet, a fluid outlet line connected to the fluid outlet, a cap, wherein the fluid inlet and the fluid outlet pass through the cap and wherein the cap occludes an end of the tumbler vial. The fluid inlet line and the fluid outlet line can be configured to twist as the tumbler vial is rotated. Other embodiments are also included herein.

This application claims the benefit of U.S. Provisional Application No. 63/705,383 filed Oct. 9, 2024, the content of which is herein incorporated by reference in its entirety.

### FIELD

Embodiments herein relate to cancer therapy systems and, more specifically, to systems for generating therapeutic suspensions of microspheres and carrier fluid that include a tumbling or rotating container.

### BACKGROUND

According to the American Cancer Society, cancer accounts for nearly 25% of the deaths that occur in the United States each year. Cancerous tumors can form if one normal cell in any part of the body mutates and then begins to grow and multiply rapidly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division.

Surgery is a common first-line therapy for many cancerous tumors. However, not every tumor can be surgically removed. Chemotherapy and immunotherapy are other common therapeutic approaches but can include substantial side effects. The use of radiation represents another approach. Specifically, radiation therapy aims at damaging the DNA of cancer cells so that they lose the capability to divide and proliferate, thus leading to the cell death process for the cancerous cells.

Brachytherapy is a form of radiation therapy where a sealed radiation source is placed inside or next to the area requiring treatment. As one form of brachytherapy, targeted radioembolization therapy can be used to treat unresectable tumors. For example, Y-90 glass microspheres can be delivered into or adjacent to a tumor through a microcatheter placed into an artery that supplies blood to the tumor. The beta radiation emitted by the Y-90 can exert a local radiotherapeutic effect on the tumor. Other radioisotopes can also be used in some types of brachytherapy.

### SUMMARY

Embodiments herein relate to cancer therapy systems generating therapeutic suspensions of microspheres and carrier fluid that include a tumbling or rotating container. In a first aspect, a cancer therapy delivery system can be included having a tumbler vial, the tumbler vial defining an interior volume. The tumbler vial can be oriented with a longitudinal axis thereof substantially horizontal with respect to gravity. The system can also include a rotation mechanism, wherein the rotation mechanism can be configured to rotate the tumbler vial. The system can also include a fluid inlet in fluid communication with the interior volume and a fluid outlet in fluid communication with the interior volume. The system can also include a fluid inlet line connected to the fluid inlet and a fluid outlet line connected to the fluid outlet. The system can also include a cap occluding an end of the tumbler vial, wherein the fluid inlet and the fluid outlet pass through the cap. The fluid inlet line and the fluid outlet line can be configured to twist as the tumbler vial is rotated.

In a second aspect, in addition to one or more of the preceding or following aspects, or in the alternative to some aspects, the tumbler vial can be oriented with the longitudinal axis thereof to be within 5 degrees of a plane perpendicular to the gravitational axis.

In a third aspect, in addition to one or more of the preceding or following aspects, or in the alternative to some aspects, the rotation mechanism can be configured to rotate the tumbler vial from 90 degrees to 720 degrees.

In a fourth aspect, in addition to one or more of the preceding or following aspects, or in the alternative to some aspects, the rotation mechanism can be configured to rotate the tumbler vial at a rotation speed of 1 to 150 rotations per minute.

In a fifth aspect, in addition to one or more of the preceding or following aspects, or in the alternative to some aspects, the rotation mechanism can be configured to rotate the tumbler vial a first direction and then a second direction opposite the first direction and then repeat the cycle indefinitely while the system is running to maintain a suspension of radioactive microspheres and carrier fluid within the tumbler vial.

In a sixth aspect, in addition to one or more of the preceding or following aspects, or in the alternative to some aspects, the rotation mechanism can be configured to rotate the tumbler vial at least 180 degrees in the first direction and at least 180 degrees in the second direction.

In a seventh aspect, in addition to one or more of the preceding or following aspects, or in the alternative to some aspects, the tumbler vial can include fins, wherein the fins can be disposed on an interior surface of the tumbler vial.

In an eighth aspect, in addition to one or more of the preceding or following aspects, or in the alternative to some aspects, the tumbler vial can be circular in cross-section.

In a ninth aspect, in addition to one or more of the preceding or following aspects, or in the alternative to some aspects, the tumbler vial can be non-circular in cross-section.

In a tenth aspect, in addition to one or more of the preceding or following aspects, or in the alternative to some aspects, the cancer therapy delivery system can further include radioactive microspheres, and a carrier fluid, wherein the radioactive microspheres and the carrier fluid can be disposed within the tumbler vial.

In an eleventh aspect, in addition to one or more of the preceding or following aspects, or in the alternative to some aspects, the carrier fluid includes an air bubble therein, wherein the air bubble can be configured to be positioned at a midpoint of the tumbler vial when the tumbler vial can be level with respect to gravity.

In a twelfth aspect, in addition to one or more of the preceding or following aspects, or in the alternative to some aspects, the radioactive microspheres have a density at least 2 times greater than the carrier fluid.

In a thirteenth aspect, in addition to one or more of the preceding or following aspects, or in the alternative to some aspects, the rotation mechanism can include a stepper motor.

In a fourteenth aspect, in addition to one or more of the preceding or following aspects, or in the alternative to some aspects, can further include a pump, wherein the pump can be in fluid communication with at least one of the fluid inlet and the fluid outlet.

In a fifteenth aspect, in addition to one or more of the preceding or following aspects, or in the alternative to some aspects, the pump can be a piston pump or a syringe pump.

In a sixteenth aspect, a cancer therapy delivery system can be included having a vial defining an interior volume, wherein the vial can be oriented with a longitudinal axis thereof substantially horizontal with respect to gravity. The system can also include a rotation mechanism, wherein the rotation mechanism can be configured to rotate the vial from 90 degrees to 720 degrees. The system can also include a plunger device, wherein the plunger device can be configured to be advanced into the interior volume of the vial. The system can also include a fluid inlet in fluid communication with the interior volume on a first side of the plunger device and a fluid outlet in fluid communication with the interior volume on a second side of the plunger device.

In a seventeenth aspect, in addition to one or more of the preceding or following aspects, or in the alternative to some aspects, the rotation mechanism can be configured to rotate the vial a first direction and then a second direction opposite the first direction and then repeat the cycle while the system is running to maintain a suspension of radioactive microspheres and carrier fluid within the vial.

In an eighteenth aspect, in addition to one or more of the preceding or following aspects, or in the alternative to some aspects, the rotation mechanism can be configured to rotate the vial at least 180 degrees in the first direction and at least 180 degrees in the second direction.

In a nineteenth aspect, in addition to one or more of the preceding or following aspects, or in the alternative to some aspects, the rotation mechanism can be configured to rotate the vial at a maximum rotation speed of 1 to 150 rotations per minute.

In a twentieth aspect, in addition to one or more of the preceding or following aspects, or in the alternative to some aspects, the cancer therapy delivery system can further include radioactive microspheres, and a carrier fluid, wherein the radioactive microspheres and the carrier fluid are disposed within the vial.

In a twenty-first aspect, in addition to one or more of the preceding or following aspects, or in the alternative to some aspects, the carrier fluid includes an air bubble therein, wherein the air bubble can be configured to be positioned at a midpoint of the vial when the vial is level with respect to gravity.

In a twenty-second aspect, in addition to one or more of the preceding or following aspects, or in the alternative to some aspects, the radioactive microspheres have a density at least 2 times greater than the carrier fluid.

In a twenty-third aspect, in addition to one or more of the preceding or following aspects, or in the alternative to some aspects, the rotation mechanism can include a stepper motor.

In a twenty-fourth aspect, in addition to one or more of the preceding or following aspects, or in the alternative to some aspects, the vial can be circular in cross-section.

In a twenty-fifth aspect, in addition to one or more of the preceding or following aspects, or in the alternative to some aspects, the vial can be non-circular in cross-section.

In a twenty-sixth aspect, a cancer therapy delivery system can be included having a suspension reservoir. The suspension reservoir can include a barrel and a plunger and can be configured to rotate. The system can also include a rotatable outlet connector, wherein the rotatable outlet connector can be in fluid communication with the suspension reservoir. The system can also include an actuator, wherein the actuator can be configured to rotate the suspension reservoir.

In a twenty-seventh aspect, in addition to one or more of the preceding or following aspects, or in the alternative to some aspects, the actuator can be configured to rotate the suspension reservoir and depress the plunger.

In a twenty-eighth aspect, in addition to one or more of the preceding or following aspects, or in the alternative to some aspects, the actuator includes a threaded portion.

In a twenty-ninth aspect, in addition to one or more of the preceding or following aspects, or in the alternative to some aspects, the cancer therapy delivery system can further include radioactive microspheres and a carrier fluid, wherein the radioactive microspheres and the carrier fluid can be disposed within the barrel.

In a thirtieth aspect, in addition to one or more of the preceding or following aspects, or in the alternative to some aspects, the radioactive microspheres have a density at least 2 times greater than the carrier fluid.

In a thirty-first aspect, in addition to one or more of the preceding or following aspects, or in the alternative to some aspects, can further include a frame member, wherein the frame member can be configured to support the suspension reservoir and the actuator.

In a thirty-second aspect, in addition to one or more of the preceding or following aspects, or in the alternative to some aspects, the system can further include an outlet tube, wherein the outlet tube can be connected to the rotatable outlet connector.

In a thirty-third aspect, in addition to one or more of the preceding or following aspects, or in the alternative to some aspects, the suspension reservoir can be configured to rotate from 90 degrees to 720 degrees.

In a thirty-fourth aspect, in addition to one or more of the preceding or following aspects, or in the alternative to some aspects, the suspension reservoir can be configured to rotate a first direction and then a second direction opposite the first direction.

In a thirty-fifth aspect, in addition to one or more of the preceding or following aspects, or in the alternative to some aspects, the suspension reservoir can include a syringe.

In a thirty-sixth aspect, in addition to one or more of the preceding or following aspects, or in the alternative to some aspects, the actuator can include a stepper motor.

In a thirty-seventh aspect, a method of making a suspension of radioactive microspheres and carrier fluid for cancer therapy can be included. The method can include putting microspheres and a carrier fluid into a tumbler vial, rotating the tumbler vial, and withdrawing a suspension of microspheres and carrier fluid from the tumbler vial through a fluid outlet and into an outlet line.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a cancer therapy delivery system in accordance with various embodiments herein.

FIG. 2 is a schematic view of a suspension generation apparatus in accordance with various embodiments herein.

FIG. 3 is a schematic cross-sectional view of a portion of a suspension generation apparatus in accordance with various embodiments herein.

FIG. 4 is a sectional view of a portion of a suspension generation apparatus in accordance with various embodiments herein.

FIG. 5 is a schematic view of a suspension generation apparatus in accordance with various embodiments herein.

FIG. 6 is a schematic cross-sectional view of a portion of a suspension generation apparatus in accordance with various embodiments herein.

FIG. 7 is a schematic cross-sectional view of a portion of a suspension generation apparatus in accordance with various embodiments herein.

FIG. 8 is a schematic view of a suspension generation apparatus in accordance with various embodiments herein.

FIG. 9 is a flowchart of operations consistent with various methods herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

The present invention relates to a cancer therapy delivery system designed to optimize the mixing of therapeutic agents, such as radioactive microspheres, with a carrier fluid to form a suspension or mixture before delivery to a patient. The system is configured to form a homogeneous mixture of the therapeutic agent and carrier fluid, thereby allowing accurate dosing of the therapeutic agent.

In various embodiments herein, the cancer therapy delivery system includes a tumbler vial, which can be a cylindrical or generally cylindrical container defining an interior volume for holding a mixture of radioactive microspheres and a carrier fluid. However, other shapes for the tumbler vial are contemplated herein as detailed below. The tumbler vial can be oriented on its side, such that a longitudinal axis thereof is substantially horizontal with respect to gravity, allowing for rotation around its longitudinal axis. This orientation can aid in mixing of the radioactive microspheres with the carrier fluid ensuring an even distribution of the microspheres within the carrier fluid.

A rotation mechanism can be connected (directly or indirectly) with the tumbler vial to facilitate its rotation. This mechanism is capable of rotating the tumbler vial. By way of example, this mechanism can rotate the tumbler vial greater than 90 degrees, such as from 90 degrees to 720 degrees, or from 180 degrees to 540 degrees, switching between a clockwise and counterclockwise direction. The rotation mechanism can operate at variable speeds, which can be adjusted according to the specific requirements of the therapy being administered. The rotation mechanism can include electrical, mechanical, or pneumatic components, or various other components. Various motors can be used with the rotation mechanism. In some embodiments, an electrical DC motor or linear ball screw can be used. In some embodiments, the inclusion of a stepper motor within the rotation mechanism allows for precise control over the rotation speed and angle, ensuring thorough mixing of the radioactive microspheres with the carrier fluid.

In some embodiments, the system is equipped with a fluid inlet and a fluid outlet, both of which pass through a cap that securely occludes one end of the tumbler vial. The fluid inlet and outlet are connected to a fluid inlet line and a fluid outlet line, respectively. These lines are designed to twist in accordance with the rotation of the tumbler vial, while preventing any kinking or obstruction of fluid flow during operation.

In some embodiments, a plunger can be included within tumbler vial and the fluid outlet can be on one side of the plunger while the fluid inlet can be on the opposite side of the plunger. Fluid entering through the fluid inlet can push the plunger through the tumbler vial expelling a mixture of the radioactive microspheres and the carrier fluid out of the fluid outlet.

In some embodiments, to enhance the mixing process, the interior surface of the tumbler vial may include fins or angled projections. These fins serve to agitate the mixture as the vial rotates, ensuring a uniform suspension of radioactive microspheres within the carrier fluid. The fins can be angled with respect to the longitudinal axis of the tumbler vial such that fluid is pushed in one direction along the longitudinal axis when the vial rotates one direction and then the fluid is pushed back in a second direction opposite the first direction when the vial rotates in a second direction that is opposite the first direction.

The system is specifically designed to work with radioactive microspheres, which are used in the treatment of various cancers. These microspheres are suspended in a carrier fluid, which is introduced into the tumbler vial. The density of the microspheres is at least two times (twice) that of the carrier fluid, necessitating the need for continuous agitation to prevent sedimentation. Further details of radioactive microspheres are provided below.

In some embodiments, an air bubble can be introduced into the carrier fluid to serve as a visual indicator of the levelness of the tumbler vial with respect to gravity. This feature is particularly useful in ensuring that the vial is oriented correctly to within 5 degrees of a plane perpendicular to the gravitational axis, thereby optimizing the mixing process.

The system can also include a pump, such as a peristaltic pump, a piston pump, a syringe pump, or another type of pump that is in fluid communication with at least one of the fluid inlet or the fluid outlet. This pump facilitates movement of the radioactive microspheres out of the tumbler vial, and out of the cancer therapy delivery system to a catheter before passing into the patient, while ensuring precise dosing and delivery of the microspheres.

The term “suspension” as used herein in reference to what embodiments of systems herein form and/or deliver as therapy to patients including carrier fluid and radioactive microspheres shall be used interchangeably with the term “mixture” unless the context dictates otherwise.

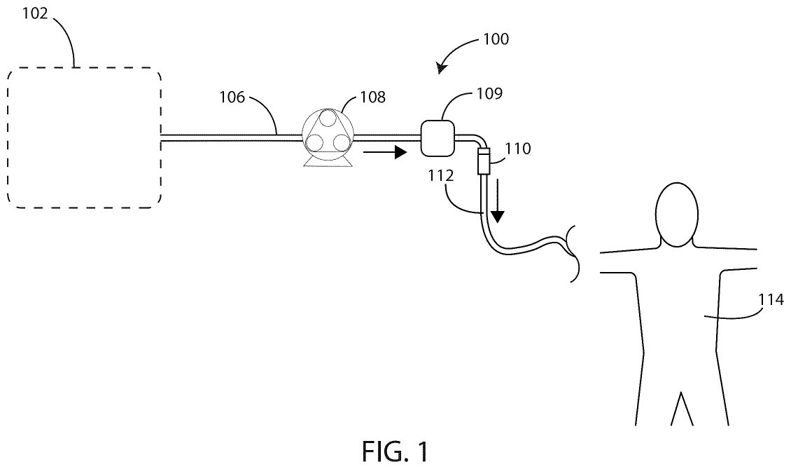

Referring now to FIG. 1, a schematic view of a cancer therapy system 100 is shown in accordance with various embodiments herein. The cancer therapy delivery system 100 is designed to optimize the delivery of therapeutic agents, such as radioactive microspheres, to a patient 114 through a highly controlled and efficient process. A key aspect of the system 100 is the suspension generation apparatus 102, which is responsible for mixing radioactive microspheres with a carrier fluid to create a homogeneous suspension. This apparatus 102 includes a tumbler vial or a suspension reservoir where the mixing process occurs. The apparatus 102 is equipped with a rotation mechanism to ensure thorough mixing by rotating the container holding the suspension. A fluid outlet line 106 is connected to the suspension generation apparatus 102 and serves as the conduit through which the prepared suspension is delivered from the apparatus to the patient 114. The fluid outlet line 106 can be formed of a polymer, such as a flexible polymer. A pump 108 is integrated into the system and is in fluid communication with the suspension generation apparatus 102 via the fluid outlet line 106 in the embodiment shown. However, it will be appreciated that a pump could also be in fluid communication with a fluid input line. As such, a pump herein can be upstream or downstream of the suspension generation apparatus 102. The pump 108 is responsible for controlling the flow rate of the suspension, ensuring that the radioactive microspheres are delivered at an optimal speed and pressure for effective treatment. In some embodiments, the system can target a flow rate of 5 to 20 milliliters per minute of the therapeutic suspension, though higher flow rates are also contemplated herein. The pump 108 can be of various types such as a peristaltic pump, a piston pump, a syringe pump, or the like.

In some embodiments, a sensor 109 can be included and can be disposed along the fluid outlet line 106 in order to track microspheres passing through and/or radiation therefrom. In some embodiments, the sensor 109 can be a physical particle counter, such as an optical particle counter. In some embodiments, the sensor 109 can be a radiation sensor, such as a beta particle counter or another type of radiation sensor. Other types of sensors are also contemplated herein, such as turbidity sensors, spectrophotometry sensors, weight sensors, and the like.

A connector 110 serves as the interface between the fluid outlet line 106 and a microcatheter 112. It ensures a secure and leak-proof connection, allowing the therapeutic suspension to be safely and efficiently delivered to the target site within the patient 114. The connector 110 can be formed of a polymeric material or another material. The microcatheter 112 is typically a slender, flexible tube that is navigated through the patient's vasculature to deliver the therapeutic suspension directly to a target site.

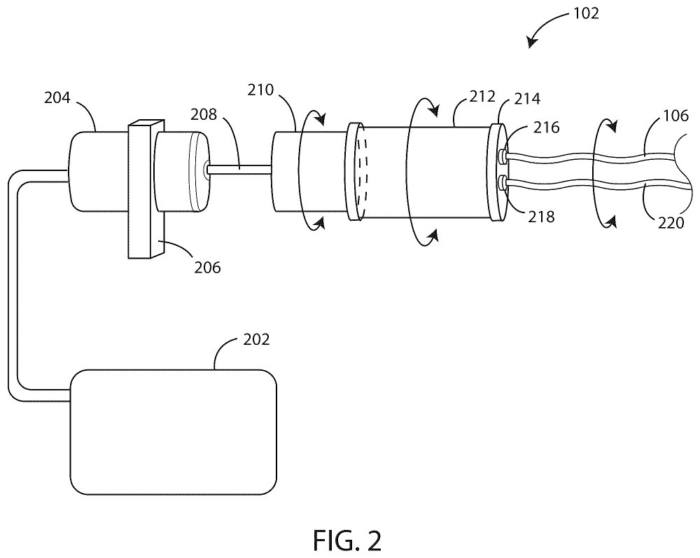

Referring now to FIG. 2, a schematic view of a suspension generation apparatus 102 is shown in accordance with various embodiments herein. The suspension generation apparatus 102 includes a tumbler vial 212. The tumbler vial 212 is a container that holds the mixture of radioactive microspheres and the carrier fluid. The tumbler vial 212 can be formed of various materials including polymers (such as an acrylic or another type of polymer), glasses, and the like and can be transparent or non-transparent. The tumbler vial 212 defines an interior volume. The size of the interior volume can vary. In some embodiments, the size of the interior volume can be from 0.25 milliliters to 50 or 100 milliliters, or larger or smaller in some applications. The tumbler vial 212 is configured to be oriented on its side during use, such as oriented with a longitudinal axis of the tumbler vial 212 substantially horizontal with respect to gravity, facilitating the mixing process. The suspension generation apparatus 102 also includes a cap 214. The cap 214 securely occludes one end of the tumbler vial 212, ensuring that the contents are contained during the mixing process.

In the embodiment of FIG. 2, both the fluid outlet 216 and the fluid inlet 218 pass through cap 214. The fluid outlet 216 and the fluid inlet 218 are in fluid communication with the interior volume of the tumbler vial 212. They enable the introduction of the carrier fluid into the vial and the subsequent extraction of the mixed suspension. The fluid outlet line 106 and fluid inlet line 220 are connected to the fluid outlet 216 and fluid inlet 218, respectively. The fluid inlet line 220 can be formed of a flexible polymer or from other materials. These lines facilitate the movement of fluids into and out of the tumbler vial 212. In various embodiments herein, the fluid lines are flexible and designed to twist as the tumbler vial is rotated, yet with sufficient rigidity to prevent any kinks or restrictions in fluid flow.

In this embodiment, generally additional carrier fluid without microspheres will flow into the tumbler vial 212 through the fluid inlet 218 as the mixed suspension of microspheres and carrier fluid exits the fluid outlet 216. As such, the concentration of microspheres to carrier fluid becomes diluted during use. However, in various embodiments herein, the system can store and/or apply a calibration curve to determine the amount of microspheres delivered per unit volume of the suspension delivered. In some embodiments, this information can be cross-referenced with data from a sensor (such as that shown in FIG. 1) in order to accurately determine the amount of microspheres that have been delivered.

The suspension generation apparatus 102 further includes rotation mechanism 204. This mechanism is configured to rotate the tumbler vial 212, ensuring the thorough mixing of the microspheres with the carrier fluid. The rotation mechanism 204 is capable of rotating the tumbler vial from 90 degrees to 720 degrees in a given direction before reversing, providing a comprehensive mixing action. The ability to rotate the vial in one direction and then the other direction in sequence repeatedly enhances mixing effectiveness, ensuring that the microspheres do not settle and remain evenly distributed within the carrier fluid.

The suspension generation apparatus 102 also includes support bracket 206 and drive shaft 208. The support bracket 206 provides structural support. In some embodiments, the support bracket 206 can be connected to other structures to aid in proper positioning of components of the suspension generation apparatus 102. By way of example, in some embodiments, the support bracket may be connected to or be part of a gyroscopic gimbal structure configured to maintain components of the suspension generation apparatus in a level position relative to the direction of gravity.

The drive shaft 208 provides a mechanical linkage for the rotation of the tumbler vial 212. The suspension generation apparatus 102 includes a rotating vial connector 210. This connector 210 attaches the tumbler vial 212 to the drive shaft 208, ensuring a secure and rotationally responsive connection. The drive shaft 208, in particular, connects to the rotating vial connector 210, translating rotational motion from the rotation mechanism 204 to the tumbler vial 212.

The suspension generation apparatus 102 of FIG. 2 also includes a control unit 202. This unit oversees the operation of the rotation mechanism 204, including the direction and speed of rotation. In various embodiments herein, the control unit 202 causes the rotation mechanism to rotate the tumbler vial a first direction and then a second direction opposite the first direction. Then, the directional rotation repeats in one direction and then the other. By way of example, in some embodiments, the control unit 202 causes the rotation mechanism to rotate the tumbler vial at least 180 degrees in the first direction and at least 180 degrees in the second direction, and then continues to switch back and forth between the first direction and the second direction. In various embodiments, the rotation mechanism 204 can be configured to rotate a tumbler vial 212 from 90 degrees to 720 degrees in a given direction before reversing. In some embodiments, the rotation mechanism 204 can be configured to rotate the tumbler vial 212 at least 180 degrees in a direction before reversing. While not intending to be bound by theory, reversing direction can beneficially nullify the buildup of centrifugal forces. In contrast, if the rotation direction never changes, the spheres can slowly work themselves towards the walls of the container inhibiting formation of a well-mixed suspension. Switching directions resets those built-up forces. Reversing direction of the rotation also acts to induce turbulence and prevent regions of stasis.

If rotational speed is too slow, then microspheres can collect at the bottom of the container inhibiting formation of a well-mixed suspension. If rotational speed is too high, then centrifugal force pushes spheres outward where they hug the walls of the container inhibiting formation of a well-mixed suspension. In various embodiments, the rotation mechanism 204 can be configured to rotate a tumbler vial 212 at rotation speeds of 1 to 150 rotations per minute (RPM) or from 10 to 75 RPM.

The suspension generation apparatus 102 also includes a motor as a part of rotation mechanism 204. In some embodiments, the rotation mechanism 204 can specifically include a stepper motor, which allows for precise control over the rotation speed and angle, ensuring the effective mixing of the suspension.

In various embodiments, the tumbler vial 212 is oriented to be within 5, 4, 3, 2, or 1 degree, or an amount falling within a range between any of the foregoing, of a plane perpendicular to the direction of gravity or gravitational axis. This can prevent the relatively heavy microspheres from being biased by gravity toward one end of the tumbler vial 212 or the other.

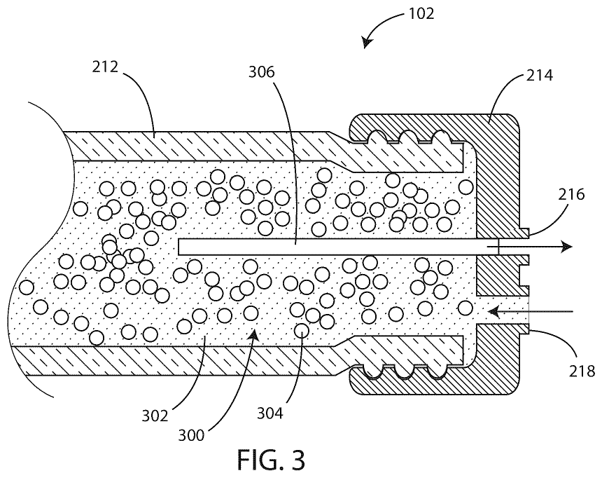

Referring now to FIG. 3, a schematic cross-sectional view of a portion of a suspension generation apparatus 102 is shown in accordance with various embodiments herein. In specific, FIG. 3 provides a schematic cross-sectional view of a portion of the suspension generation apparatus 102, specifically focusing on a tumbler vial 212 and its internal components, as utilized in the cancer therapy delivery system. This figure illustrates aspects of an internal arrangement designed to facilitate the preparation of a therapeutic suspension comprising radioactive microspheres and a carrier fluid.

The suspension generation apparatus 102 includes tumbler vial 212. This typically cylindrical container is shown in cross-section, revealing an interior volume 300 where the suspension of radioactive microspheres 304 in the carrier fluid 302 is prepared. FIG. 3 also shows the cap 214, which is positioned on a side of the tumbler vial 212. The cap 214 securely seals the container, preventing any leakage or contamination. Through this cap, both the fluid outlet 216 and the fluid inlet 218 are integrated. The fluid outlet 216 and fluid inlet 218 provide pathways for the carrier fluid 302 to enter the interior volume 300 of the tumbler vial 212 where it is mixed with radioactive microspheres 304 forming a suspension which then passes out of the fluid outlet 216 for delivery to the patient.

The interior volume 300 within the tumbler vial 212, where the mixing of the radioactive microspheres 304 with the carrier fluid 302 occurs, is configured to accommodate the necessary quantities of both components to achieve the desired therapeutic effect. The carrier fluid 302 serves as the medium in which the radioactive microspheres 304 are suspended. It facilitates delivery of the microspheres when administered to the patient. The carrier fluid 302 is typically an aqueous solution, such as a saline solution.

In various embodiments, a fluid outlet projection 306 is included. The fluid outlet projection 306 can be a tube or needle-like structure that extends into the interior volume 300 from the fluid outlet 216, designed to optimize the extraction of the suspension, ensuring that the therapeutic mixture can be efficiently drawn out for delivery. The fluid outlet projection 306 can ensure that extraction of the suspension takes place at a point that is spaced apart from the fluid inlet 218, so that only a thoroughly mixed suspension is extracted. As described further below, the radioactive microspheres 304 have a density at least two times greater than that of the carrier fluid 302. This characteristic is significant for the design and operation of the suspension generation apparatus 102, as it influences the mixing dynamics within the tumbler vial 212. The difference in density necessitates the thorough mixing provided by the apparatus to ensure that the microspheres are evenly suspended within the carrier fluid, preventing sedimentation and ensuring a homogeneous therapeutic suspension for effective cancer treatment.

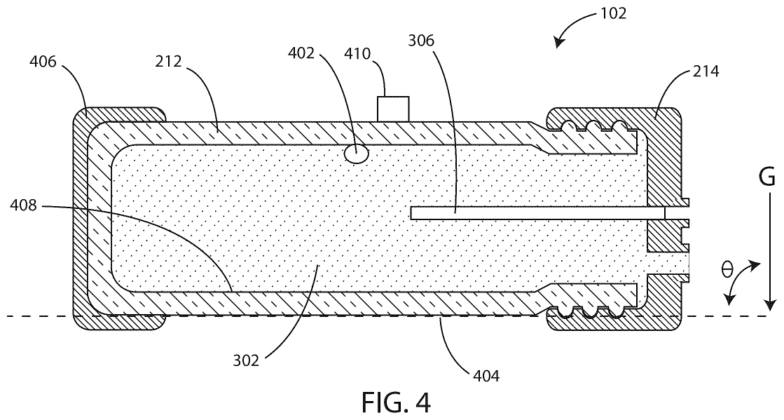

Referring now to FIG. 4, a sectional view of a portion of a suspension generation apparatus 102 is shown in accordance with various embodiments herein. In specific, FIG. 4 provides a sectional view of a portion of the suspension generation apparatus 102, focusing on the tumbler vial 212 and its interaction with the carrier fluid 302 and an air bubble 402. As before, the suspension generation apparatus 102 includes tumbler vial 212. This is the container where the carrier fluid 302 and radioactive microspheres (not shown in this view) are mixed. Further, the cap 214 is shown sealing the tumbler vial 212 and ensuring that the contents are securely contained during the mixing process. Further, the fluid outlet projection 306 extends into the interior of the vial from the cap 214.

In this view, an air bubble 402 is shown positioned within the carrier fluid 302. The air bubble serves as an indicator of the levelness and orientation of the tumbler vial 212 with respect to the direction of gravity. The air bubble's 402 position within the carrier fluid provides visual feedback regarding the vial's alignment with respect to the direction of gravity. In various embodiments, the vial 212 is configured so that the air bubble 402 is positioned at a midpoint of the tumbler vial 212 when the vial 212 is level with respect to gravity. In some embodiments, an air bubble sensor can be mounted on an inside or an outside surface of the tumbler vial.

FIG. 4 also shows bottom surface 404, which is the lowermost surface of the tumbler vial 212 when it is placed in its operational orientation on its side. The bottom surface 404 is parallel with the bottom inner surface 408 of the tumbler vial 212. To promote ideal mixing, it is optimal for the bottom inner surface 408 to be as close to perpendicular to the direction of the gravity as possible. In various embodiments herein, the orientation of the lengthwise axis of the tumbler vial 212 (which is parallel with the bottom inner surface 408) is within 5 degrees of a plane that is perpendicular to the gravitational axis. Such an orientation is beneficial for ensuring the effective mixing of the carrier fluid 302 and the radioactive microspheres.

In some embodiments, a level sensor 410 can be included to aid in ensuring that the tumbler vial 212 is sufficiently level. In some embodiments, the level sensor 410 can include an accelerometer and/or a gyroscopic sensor to detect the degree to which the tumbler vial 212 is level. While not shown in this view, in some embodiments, the system can include a level adjustment device, such as a level adjustment screw that changes the level position of the tumbler vial 212 relative to other components such as a frame (described further below) to allow a system user to adjust the tumbler vial 212 angle to make it sufficiently level.

In various embodiments, a vial receiving cover 406 can be placed over an end of the tumbler vial 212 on an end that is opposite the cap 214. The vial receiving cover 406 can support and secure the tumbler vial 212 in its operational position. It can also facilitate attachment of the tumbler vial 212 with rotating vial connector 210 (shown in FIG. 2).

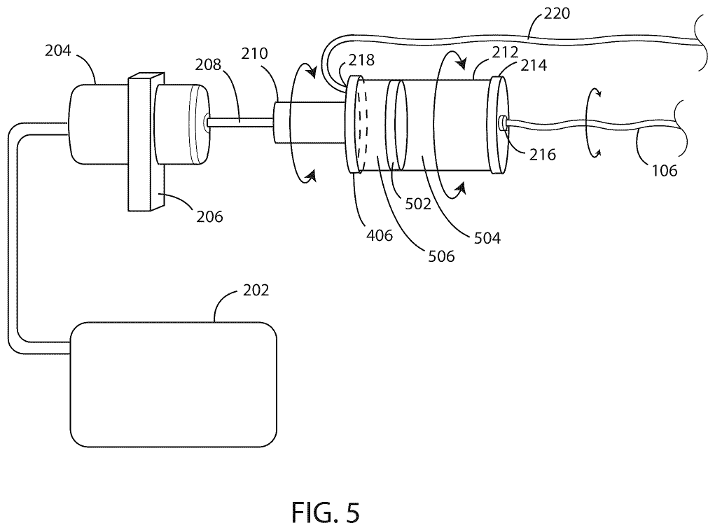

Referring now to FIG. 5, a schematic view of a suspension generation apparatus 102 is shown in accordance with various embodiments herein. In specific, FIG. 5 presents a schematic view of a suspension generation apparatus 102, showcasing another embodiment designed to enhance the preparation and delivery of therapeutic suspensions, such as those containing radioactive microspheres in a carrier fluid, for cancer therapy. This figure specifically illustrates the integration of a plunger device within the apparatus, highlighting its role in the controlled manipulation of the suspension's volume and pressure.

As before, FIG. 5 shows a tumbler vial 212 where the carrier fluid and radioactive microspheres are mixed to create the therapeutic suspension, a cap 214 that seals the tumbler vial 212, and a fluid outlet 216 and a fluid inlet 218, providing pathways for the introduction and extraction of the suspension components. FIG. 5 also shows a fluid outlet line 106 connected to the fluid outlet 216, which facilitates the movement of the mixed suspension from the tumbler vial to the patient. FIG. 5 also shows rotation mechanism 204, support bracket 206, drive shaft 208, and rotating vial connector 210. These components collectively enable the rotation of the tumbler vial 212, ensuring thorough mixing of the microspheres with the carrier fluid. Further, FIG. 5 shows control unit 202, which directs the operation of the rotation mechanism 204, including the direction and speed of rotation, and vial receiving cover 406 which supports and secures the tumbler vial 212 in its operational position, and facilitates connection of the vial 212 to the rotation vial connector 210.

However, in contrast to the embodiment of FIG. 2, FIG. 5 also shows a plunger device 502. The plunger device 502 is configured to be advanced into the interior volume of the tumbler vial 212. The interior volume of the tumbler vial 212 is divided into a first portion 504 and a second portion 506, with the first portion 504 located on one side of the plunger device 502 and the second portion 506 on the opposite side. In this embodiment, the fluid inlet 218 is disposed on the vial receiving cover 406 and connects to a fluid inlet line 220. As a fluid (which could be a liquid such as carrier fluid or a gas) is pushed from fluid inlet line 220 and through fluid inlet 218, the pressure increases in the second portion 506 of the interior volume of the tumbler vial 212 causing the plunger device 502 to move and contract the size of first portion 504 expelling suspension through the fluid outlet 216 and into fluid outlet line 106. In summary, the inclusion of the plunger device 502, which can be advanced into the interior volume of the vial, represents an innovative approach to managing the fluid dynamics within the vial. The fluid inlet 218 and fluid outlet 216 are in fluid communication with the interior volume on opposite sides of the plunger device, ensuring that the therapeutic suspension can be introduced, mixed, and extracted efficiently. While not intending to be bound by theory, the embodiment of FIG. 5 with a plunger can be advantageous as it allows for consistent concentrations of microspheres in the carrier fluid throughout the dosing process because the side of the interior volume of the tumbler vial 212 does not become diluted with new carrier fluid during the suspension generation and delivery.

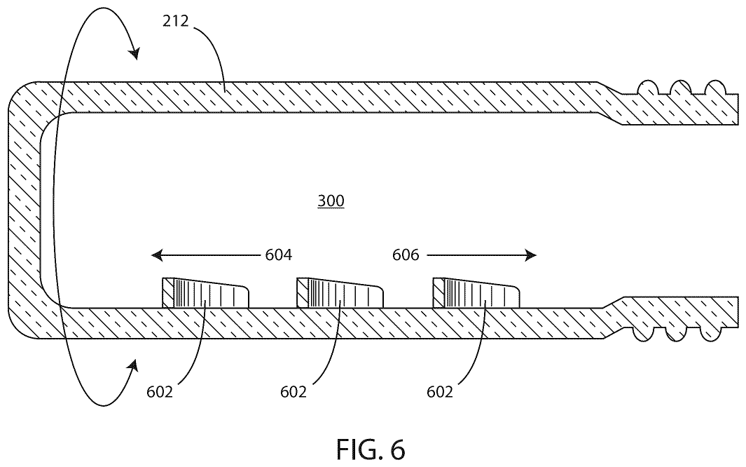

Referring now to FIG. 6, a schematic cross-sectional view of a portion of a suspension generation apparatus is shown in accordance with various embodiments herein. In specific, FIG. 6 provides a schematic cross-sectional view of a portion of the suspension generation apparatus, specifically focusing on the interior of the tumbler vial 212. This figure highlights possible internal features within the tumbler vial that can aid in the effective mixing of therapeutic agents, such as radioactive microspheres, with a carrier fluid for cancer therapy applications. FIG. 6 shows tumbler vial 212, which is the primary container where the mixing of the radioactive microspheres and the carrier fluid occurs, creating a homogeneous therapeutic suspension. The tumbler vial 212 is shown in cross-section in this view and illustrates the interior volume 300 thereof.

In this embodiment, fins 602 are disposed on the interior surface of the tumbler vial 212. These fins 602 can be useful for the mixing process, as they create turbulence as the tumbler vial 212 rotates and can facilitate the thorough integration of the microspheres within the carrier fluid when the vial is rotated. The fins 602 can be angled with respect to the longitudinal axis of the tumbler vial 212 such that when the tumbler vial 212 is rotated in a first direction, the fluid inside the tumbler vial 212 is pushed in a first direction 604 and when the tumbler vial is rotated in the opposite direction then the fluid inside the tumbler vial 212 is pushed in a second direction 606. Thus, the fins 602 can improve the mixing efficiency of the suspension generation apparatus by creating turbulence within the carrier fluid as the tumbler vial is rotated, ensuring that the radioactive microspheres are evenly suspended, preventing sedimentation and promoting a homogeneous therapeutic suspension. The fins 602 can be attached to the tumbler vial 212 using various techniques including adhesive bonding, mechanical attachment, integral molding, and the like. In some cases, the fins 602 can be made of the same material as the tumbler vial 212 (such as a glass or polymer) and in some can be made of a different material.

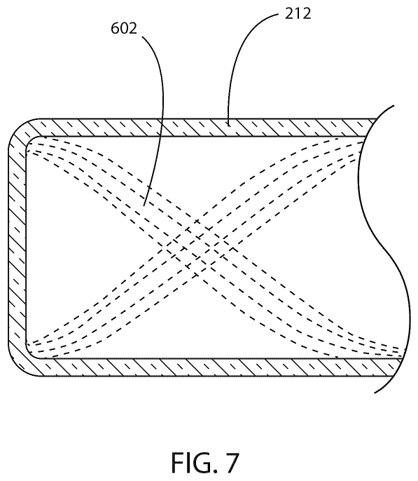

Beyond fins, other types of structures or surface features can be disposed on the inner surface of the tumbler vial to enhance mixing action. By way of example, ridges, projections, bumps, paddles, and the like, or even surface roughness can be used to enhance mixing action within the tumbler vial. In some embodiments, the fins 602 can wrap radially around the entire interior surface of the tumbler vial 212. For example, FIG. 7 shows a schematic cross-sectional view of a portion of a suspension generation apparatus including a tumbler vial 212 and fins 602 therein that wrap radially around the entire interior surface of the tumbler vial 212. In some embodiments, the fins 602 can be configured in a helical pattern.

In various embodiments, the tumbler vial can have a cross-section that is circular. However, in some embodiments, a tumbler vial can have a cross-section that is not circular. By way of example, in some embodiments, a tumbler vial herein can have a cross-section that is elliptical, star-shaped, irregular, or have other radial geometry. Such added radial geometry can aid in the mixing or formation of the suspension. In some embodiments, the radial geometry of the tumbler vial can be non-circular, but also lack corners or edges.

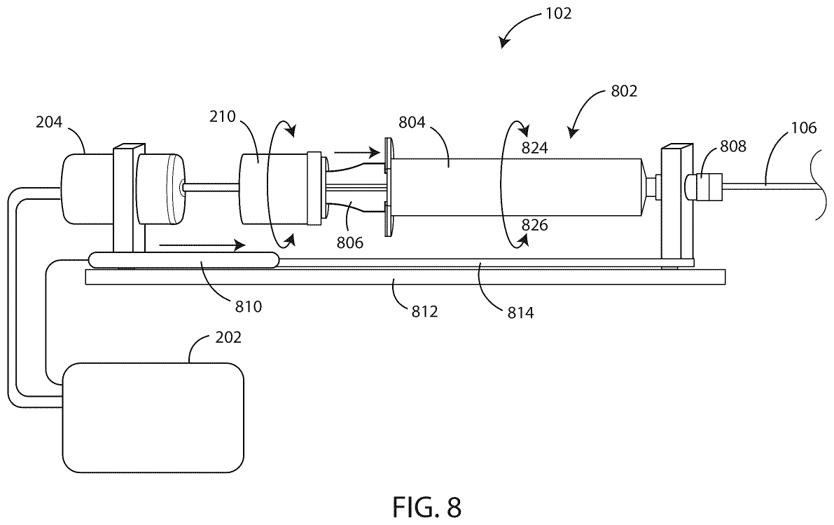

Referring now to FIG. 8, a schematic view of a suspension generation apparatus 102 is shown in accordance with various embodiments herein. In specific, FIG. 8 illustrates a schematic view of a suspension generation apparatus, showcasing an embodiment configured to facilitate the preparation and delivery of therapeutic suspensions for cancer therapy. Similar to other embodiments, FIG. 8 shows a control unit 202, a fluid outlet line 106, a rotation mechanism 204, and a rotating vial connector 210.

However, FIG. 8 also shows a number of features distinct from other embodiments. FIG. 8 shows a suspension reservoir 802, which is designed to hold and mix the therapeutic suspension (radioactive microspheres and carrier fluid). In various embodiments, the suspension reservoir 802 can be configured to rotate. In various embodiments, the suspension reservoir 802 is configured to rotate from 90 degrees to 720 degrees. In various embodiments, the suspension reservoir 802 is configured to rotate a first direction 824 and then a second direction 826 opposite the first direction 604.

The suspension reservoir 802 can include a barrel 804 and a plunger 806, which work together to contain and manipulate the suspension. In some embodiments, the suspension reservoir 802 can take the form of a syringe. The barrel 804 is the main body of the suspension reservoir 802, where the therapeutic suspension, including radioactive microspheres and carrier fluid, is stored and mixed. The plunger 806 is a movable component within the barrel 804. The plunger 806 is used to adjust the volume and pressure of the suspension within the reservoir 802, facilitating the mixing process and the controlled delivery of the suspension.

FIG. 8 also shows a rotatable outlet connector 808. This feature allows for the connection of the suspension reservoir 802 to the fluid outlet line 106, enabling the rotation of the reservoir without disrupting the fluid connection. In some embodiments, the rotatable outlet connector 808 can take the form of a rotating Luer lock connector.

Some components of the system can move linearly in order to facilitate depression of the plunger 806. For example, a movement actuator 810 (which can include an electric motor or another type of actuator) can be included and can be configured to move along a rail 814 associated with the frame 812. The frame member 812 holds the various components in place, while the rail 814 facilitates the precise movement and positioning of the actuator 810. The movement actuator 810 can carry the rotation mechanism 204 and as the rotating mechanism 204 (attached to the plunger 806 directly or indirectly) moves relative to the barrel 804, the plunger 806 can move farther into the barrel 804. Depression of the plunger 806 can occur simultaneously with rotation of the suspension reservoir 802. In some embodiments, depression of the plunger 806 can be effectuated in other ways. The embodiment of FIG. 8 can be advantageous as the suspension reservoir 802 including the barrel 804 and the plunger 806 can be easily separated from other components of the system and are formed to be disposable (such as formed from a polymer) allowing of easy and safe disposal of material that has come into contact with radioactive material after a delivery procedure has been completed.

### Methods

Many different methods are contemplated herein, including, but not limited to, methods of making and/or delivering suspensions/mixtures of microspheres and carrier fluid for cancer therapy, methods of using suspensions/mixtures of microspheres and carrier fluid for cancer therapy, and the like. Aspects of system/device operation described elsewhere herein can be performed as operations of one or more methods in accordance with various embodiments herein.

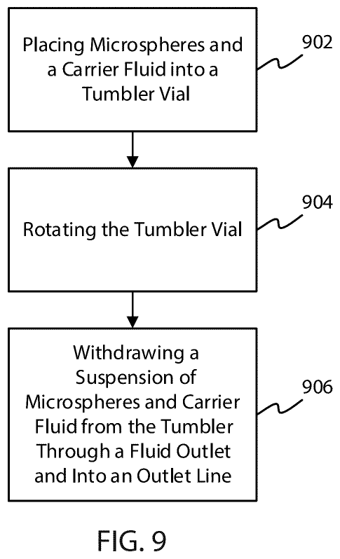

In an embodiment, a method of making a suspension of microspheres and carrier fluid for cancer therapy is included. Referring now to FIG. 9, the method can include an operation of placing 902 microspheres and a carrier fluid within a tumbler vial. The method can further include rotating 904 the tumbler vial. The method can further include rotating the tumbler vial in a first direction and then back in a second direction that is opposite the first direction and then repeating to switch between the first direction and the second direction continuously. The method can further include withdrawing 906 a suspension of microspheres and carrier fluid from the tumbler vial through a fluid outlet and into an outlet line. The method can further include conveying the suspension from the outlet line and into a catheter. In some embodiments, the method can include twisting a fluid inlet line and a fluid outlet line that are connected to a cap for the tumbler vial as the tumbler vial rotates back and forth. In some embodiments, the method can include inserting fluid into a space within the tumbler vial on a first side of a plunger and withdrawing a suspension of microspheres and carrier fluid from a space within the tumbler vial on a second side of the plunger.

### Microspheres

Microspheres herein can include those with a combination of yttria, alumina, and silica. By way of example, in some embodiments, microspheres herein can include Y₂O₃—Al₂O₃—SiO₂ in a 40:20:40 wt. % ratio. It will be appreciated, however, that other types of microspheres are also contemplated herein including those with other materials.

In some embodiments, microspheres can be prepared by combining yittrium-89 with alumina and silica, in some cases also using a flame spheroidization method, and using neutron bombardment to convert Y-89 into the beta emitting radioisotope Y-90. In various embodiments, the amount of beta radiation can exceed 400, 600, 800, 1000, 1500, 2000, 2500, 3000, 4000, 5000, 6000, 7000, 8000, 9000, 10000, or even 12000 Becquerel (Bq) per sphere or higher at the time of activity calibration (recognizing that the amount of radiation will drop after that point as the Y-90 radioisotope decays). In some embodiments, the microspheres can be provided in a vial with activity of 3 Gigabecquerel (GBq) or lower up to 20 GBq or higher (at calibration time or “reference date and time”). However, in some embodiments, the microspheres can be provided in a vial with activity of less than 3, 2.75, 2.5, 2.25, 2, 1.75, 1.5, 1.25, 1.0, 0.75, 0.5, 0.4, 0.3, 0.35, 0.2, 0.15, 0.1, 0.05, or 0.01 GBq, or less at calibration time, or an amount falling within a range between any of the foregoing. It will be appreciated that the use of other isotopes is also contemplated herein.

It will be appreciated that dosages can vary based on factors including the type of tumor/tissue to be treated, location of the tumor/tissue to be treated, factors specific to a particular patient, and the like. In some embodiments the dosage of the therapy can be less than or equal to 5000 Gray (Gy), 4500 Gy, 4000 Gy, 3500 Gy, 3000 Gy, 2500 Gy, 2000 Gy, 1500 Gy, 100 Gy, 500 Gy, 400 Gy, 300 Gy, 250 Gy, 225 Gy, 200 Gy, 180 Gy, 150 Gy, 120 Gy, 100 Gy, 90 Gy, 80 Gy, 70 Gy, 60 Gy, 50 Gy, 40 Gy, 30 Gy, or 20 Gy, or an amount falling within a range between any of the foregoing.

The size of the microspheres can be extremely small. In some embodiments, the average diameter of the microspheres can be from about 15 micrometers (μm) to about 35 μm. However, in some embodiments the microspheres can be somewhat smaller or larger.

The density of the microspheres can be quite high. In some embodiments, the density of the microspheres can be above 1.4, 1.8, 2, 2.5, or 3 grams/milliliter (g/mL), such as from 3.1 to 3.5 g/mL, or about 3.3 g/mL. By comparison, the density of water at room temperature is about 0.9978 g/mL. As such, the density of microspheres is much higher than an exemplary carrier fluid such as a saline solution which influences how readily such microspheres can settle out of a suspension.

It should be noted that, as used in this specification and the appended claims, the singular forms “a,” “an,” and “the” include plural referents unless the content clearly dictates otherwise. It should also be noted that the term “or” is generally employed in its sense including “and/or”unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A cancer therapy delivery system comprising:
a tumbler vial, the tumbler vial defining an interior volume;
wherein the tumbler vial is oriented with a longitudinal axis thereof substantially horizontal with respect to gravity;
a rotation mechanism, wherein the rotation mechanism is configured to rotate the tumbler vial;
a fluid inlet, wherein the fluid inlet is in fluid communication with the interior volume;
a fluid outlet, wherein the fluid outlet is in fluid communication with the interior volume;
a fluid inlet line, wherein the fluid inlet line is connected to the fluid inlet;
a fluid outlet line, wherein the fluid outlet line is connected to the fluid outlet;
a cap;
wherein the fluid inlet and the fluid outlet pass through the cap;
wherein the cap occludes an end of the tumbler vial; and
wherein the fluid inlet line and the fluid outlet line are configured to twist as the tumbler vial is rotated.

2. The cancer therapy delivery system of claim 1, wherein the tumbler vial is oriented with the longitudinal axis thereof to be within 5 degrees of a plane perpendicular to the gravitational axis.

3. The cancer therapy delivery system of claim 1, wherein the rotation mechanism is configured to rotate the tumbler vial from 90 degrees to 720 degrees.

4. The cancer therapy delivery system of claim 1, wherein the rotation mechanism is configured to rotate the tumbler vial at a rotation speed of 1 to 150 rotations per minute.

5. The cancer therapy delivery system of claim 1, wherein the rotation mechanism is configured to rotate the tumbler vial a first direction and then a second direction opposite the first direction and then repeat the cycle while the system is running to maintain a suspension of radioactive microspheres and carrier fluid within the tumbler vial.

6. The cancer therapy delivery system of claim 5, wherein the rotation mechanism is configured to rotate the tumbler vial at least 180 degrees in the first direction and at least 180 degrees in the second direction.

7. The cancer therapy delivery system of claim 1, the tumbler vial comprising fins, wherein the fins are disposed on an interior surface of the tumbler vial.

8. The cancer therapy delivery system of claim 1, wherein the tumbler vial is non-circular in cross-section.

9. The cancer therapy delivery system of claim 1, further comprising:
radioactive microspheres; and
a carrier fluid, wherein the radioactive microspheres and the carrier fluid are disposed within the tumbler vial.

10. The cancer therapy delivery system of claim 9, wherein the carrier fluid includes an air bubble therein, wherein the air bubble is configured to be positioned at a midpoint of the tumbler vial when the tumbler vial is level with respect to gravity.

11. A cancer therapy delivery system comprising:
a vial, the vial defining an interior volume;
wherein the vial is oriented with a longitudinal axis thereof substantially horizontal with respect to gravity;
a rotation mechanism, wherein the rotation mechanism is configured to rotate the vial from 90 degrees to 720 degrees;
a plunger device, wherein the plunger device is configured to be advanced into the interior volume of the vial;
a fluid inlet, wherein the fluid inlet is in fluid communication with the interior volume on a first side of the plunger device; and
a fluid outlet, wherein the fluid outlet is in fluid communication with the interior volume on a second side of the plunger device.

12. The cancer therapy delivery system of claim 11, wherein the rotation mechanism is configured to rotate the vial a first direction and then a second direction opposite the first direction and then repeat the cycle while the system is running to maintain a suspension of radioactive microspheres and carrier fluid within the vial.

13. The cancer therapy delivery system of claim 12, wherein the rotation mechanism is configured to rotate the vial at least 180 degrees in the first direction and at least 180 degrees in the second direction.

14. The cancer therapy delivery system of claim 11, further comprising:
radioactive microspheres; and
a carrier fluid, wherein the radioactive microspheres and the carrier fluid are disposed within the vial.

15. The cancer therapy delivery system of claim 14, wherein the carrier fluid includes an air bubble therein, wherein the air bubble is configured to be positioned at a midpoint of the vial when the vial is level with respect to gravity.

16. The cancer therapy delivery system of claim 14, wherein the radioactive microspheres have a density at least 2 times greater than the carrier fluid.

17. The cancer therapy delivery system of claim 11, the rotation mechanism comprising a stepper motor.

18. The cancer therapy delivery system of claim 11, wherein the vial is circular in cross-section.

19. The cancer therapy delivery system of claim 11, wherein the vial is non-circular in cross-section.

20. A method of making a suspension of radioactive microspheres and carrier fluid for cancer therapy comprising:
putting microspheres and a carrier fluid into a tumbler vial;
rotating the tumbler vial; and
withdrawing a suspension of microspheres and carrier fluid from the tumbler vial through a fluid outlet and into an outlet line.
